(19) United States
(12) Patent Application Publication
(10) Pub. No.: US 2009/0157085 A1
(43) Pub. Date: Jun. 18, 2009

(54) DEVICE AND METHOD FOR INTRODUCING A BONE CEMENT MIXTURE INTO A DAMAGED BONE

(75) Inventor: Jeffry S. Melsheimer, Springville, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: COOK INCORPORATED, BLOOMINGTON, IN (US)

(21) Appl. No.: 11/958,903

(22) Filed: Dec. 18, 2007

(51) Int. Cl. A61B 17/58; A61M 5/00

(52) U.S. Cl. 606/93; 606/92; 604/264

(57) ABSTRACT

In at least one embodiment of the present invention, a device for introducing a bone cement mixture into a damaged bone of a patient is provided. The device comprises a needle including a cannula and a tip portion extending therefrom. A lumen is formed through the cannula and the cannula has a distal portion. The lumen is for advancing the bone cement mixture to the distal portion. The distal portion has an open distal end and a side aperture formed through the distal portion. Attached to the distal portion of the cannula is the tip portion. The tip portion is configured for piercing the damaged bone to define a bone opening and to direct advancement of the bone cement mixture towards the side aperture while preventing advancement of the bone cement mixture through the distal end of the distal portion of the cannula.

### BACKGROUND OF THE INVENTION

1. Field of the Invention

The present invention relates to a device, a kit and a method for introducing a bone cement mixture into a damaged bone of a patient.

2. Background

There is a clinical need to fill and stabilize damaged bones of patients, such as for example, filling defects and collapsed vertebra of patients suffering from severe back pain caused by osteoporosis, metastatic tumors or back injuries. Currently, these defects are repaired using multi-component bone cements that are mixed in open containers, transferred to a device and injected from the device into the damaged bone where the mixture chemically reacts or cures to form a solid structure.

The most widely used bone cements are based on polymethylmethacrylate (PMMA) and hydroxyapatite. These materials have relatively good strength characteristics, but have a number of drawbacks. These cements are a two-part chemically reactive system and have approximately 5-10 minutes of working time once the components are mixed together to form a bone cement mixture. The bone cement mixture is, for example, then injected into a collapsed vertebra of a patient, typically with a syringe in fluid communication with a large bore needle that has been inserted into the vertebra. Once the vertebra is filled with the bone cement mixture, the large bore needle is retracted from the vertebra. Several problems however can occur with this procedure.

One problem is that the large bore needle may continue to advance the highly viscous bone cement mixture through its open distal end while the needle is being retracted from the vertebra, leaving remnants of the bone cement mixture attached to the vertebra. These remnants cure, becoming solid structures extending from the vertebra. Some of the solidified remnants may be long and protrude from the body, or in other cases, they may be short making them difficult to access. In either case, the remnants are problematic for the patient and often, they must be removed. Removal of the remnants, however, can sometimes be difficult to do.

Another problem is that the bone cement mixture cures prior to the large bore needle being fully retracted from the vertebra. In this scenario, the needle may become affixed to the vertebra. Specifically, the solidified bone cement mixture disposed in the needle is bonded to the solidified bone cement mixture that fills the vertebra. Because of the high tensile strength of the solidified bone cement mixture, the bone cement bond may be difficult to break or “snap apart” by pulling and/or twisting the needle.

### BRIEF SUMMARY OF THE INVENTION

Embodiments of the present invention provide a device, a kit and a method that may significantly reduce the likelihood of bone cement remnants being left on a damaged bone of a patient by a retracting needle used to fill the bone with a bone cement mixture. Moreover, some of these embodiments may facilitate breaking a bone cement bond formed between the filled bone and the needle when the bone cement mixture cures prior to the needle being fully retracted from the bone.

In at least one embodiment of the present invention, a device for introducing a bone cement mixture into a damaged bone of a patient is provided. The device comprises a needle including a cannula and a tip portion extending from the cannula. A lumen is formed through the cannula and the cannula has a distal portion. The lumen is for advancing the bone cement mixture to the distal portion. The distal portion has an open distal end and a side aperture formed through the distal portion. Attached to the distal portion of the cannula is the tip portion. Proximal the tip portion is the side aperture. The tip portion is configured for piercing the damaged bone of the patient to define a bone opening and to direct advancement of the bone cement mixture towards the side aperture while preventing advancement of the bone cement mixture through the distal end of the distal portion of the cannula. The distal portion is configured to tightly fit the bone opening such that advancement of the cement mixture through the side aperture is closed-off when the distal portion is retracted through the bone opening.

In at least one other embodiment of the present invention, a bone cement substitute kit for introducing a bone cement mixture into the damaged bone of a patient is provided. The kit comprises a first bone cement component and a second bone cement component. The first and second bone cement components form the bone cement mixture. An injection device is for containing the bone cement mixture and includes an outlet. The injection device is configured to dispense the bone cement mixture from the injection device by advancing the bone cement mixture through the outlet. In fluid communication with the outlet is a needle. The needle includes a cannula and a tip portion extending therefrom. Formed through the cannula is a lumen. The cannula has a distal portion. The lumen is for advancing the bone cement mixture to the distal portion. The distal portion has an open distal end and a side aperture formed through the distal portion. Attached to the distal portion of the cannula is the tip portion. Proximal the tip portion is the side aperture. The tip portion is configured for piercing the damaged bone of the patient to define a bone opening and to direct advancement of the bone cement mixture towards the side aperture while preventing advancement of the bone cement mixture through the distal end of the distal portion of the cannula. The distal portion is configured to tightly fit the bone opening such that advancement of the bone cement mixture through the side aperture is closed-off when the distal portion is retracted through the bone opening.

In at least one other embodiment of the present invention, a method for introducing a bone cement mixture into a damaged bone of a patient is provided. The method comprises mixing a first bone cement component together with a second bone cement component to form the bone cement mixture. The bone cement mixture is contained in an injection device. A needle is inserted into the damaged bone of the patient. The needle includes a cannula and a tip portion extending from the cannula. Formed through the cannula is a lumen and the cannula has a distal portion. The distal portion has an open distal end and a side aperture formed through the distal portion. The tip portion is attached to the distal portion of the cannula such that the side aperture is proximal the tip portion. The tip portion pierces the damaged bone to define a bone opening. The distal portion is configured to tightly fit the bone opening. Fluid communication between an outlet of the injection device and the lumen of the cannula is provided. The bone cement mixture is dispensed from the injection device by advancing the bone cement mixture through the outlet. The bone cement mixture is introduced to the damaged bone of the patient which includes advancing the bone cement mixture through the lumen of the needle. The tip portion directs advancement of the bone cement mixture through the side aperture while preventing advancement of the bone cement mixture through the distal end of the needle. The damaged bone of the patient is filled with the bone cement mixture. The needle is removed from the damaged bone by at least one of a pulling motion and a twisting motion which closes off advancement of the bone cement mixture through the side aperture when the distal portion is retracted through the bone opening.

Further objects, features and advantages of the invention will become apparent from consideration of the following description and the appended claims when taken in connection with the accompanying drawings.

### BRIEF DESCRIPTION OF THE DRAWINGS

FIG. 1 is a perspective view of a collapsed vertebra;

FIG. 2 is a partial side view of a device for stabilizing a collapsed vertebra in accordance with one embodiment of the present invention;

FIG. 3 is an enlarged view of FIG. 2;

FIG. 4 is a partial side view of a device for stabilizing a collapsed vertebra in accordance with yet another embodiment of the present invention;

FIG. 5 is a partial side view of a stabilized collapsed vertebra in accordance with one example of the present invention;

FIG. 6 is a perspective view of a device for stabilizing a collapsed vertebra in accordance with one embodiment of the present invention;

FIG. 7a is an enlarged partial side view of the device for stabilizing a collapsed vertebra in accordance with one example of the present invention;

FIG. 7b is an enlarged partial side view of the device for stabilizing a collapsed vertebra in accordance with another example of the present invention;

FIG. 7c is an enlarged partial side view of the device for stabilizing a collapsed vertebra in accordance with yet another example of the present invention;

FIG. 7d is an enlarged partial side view of the device for stabilizing a collapsed vertebra in accordance with another example of the present invention;

FIG. 7e is an enlarged partial side view of the device for stabilizing a collapsed vertebra in accordance with yet another example of the present invention;

FIG. 7f is an enlarged partial side view of the device for stabilizing a collapsed vertebra in accordance with another example of the present invention;

FIG. 7g is an enlarged partial side view of the device for stabilizing a collapsed vertebra in accordance with yet another example of the present invention;

FIG. 7h is an enlarged partial side view of the device for stabilizing a collapsed vertebra in accordance another example of the present invention;

FIG. 7i is an enlarged partial side view of the device for stabilizing a collapsed vertebra in accordance with yet another example of the present invention;

FIG. 7j is an enlarged side view of the device for stabilizing a collapsed vertebra in accordance with another example of the present invention;

FIG. 8 is a perspective view of a device for stabilizing a collapsed vertebra in accordance with another embodiment of the present invention;

FIG. 9 is a side view of a bone cement substitute kit in accordance with one embodiment of the present invention;

FIG. 10 is an exploded view of a bone substitute kit in accordance with another embodiment of the present invention; and

FIG. 11 is a flow chart for a method for introducing a bone cement mixture into a damaged bone of a patient in accordance with an embodiment of the present invention.

### DETAILED DESCRIPTION OF THE INVENTION

Detailed embodiments of the present invention are disclosed herein. It is understood however, that the disclosed embodiments are merely exemplary of the invention and may be embodied in various and alternative forms. The figures are not necessarily to scale; some figures may be configured to show the details of a particular component. Therefore, specific structural and functional details disclosed herein are not to be interpreted as limiting but merely as a representative basis with the claims and for teaching one skilled in the art to practice the present invention.

Examples of the present invention seek to overcome some of the concerns associated with stabilizing the damaged bone of a patient with a bone cement mixture while minimizing the likelihood of leaving remnants of the bone cement mixture attached to the bone and/or facilitating removal of a needle that has become inadvertently affixed to the bone during such a procedure.

Employing the principles of the present invention is, for example, a device, a kit and a method for introducing a bone cement mixture into a damaged bone of a patient. The device, which is utilized in both the kit and the method, includes a needle that is used for introducing the bone cement mixture into the damaged bone. The needle dispenses the bone cement mixture through a side aperture formed in a portion of the needle which tightly fits a bone opening created by the needle. The needle pierces the bone, forming a substantially cylindrical wall through a portion of the bone to define the bone opening. When the needle is retracted through the bone opening, the wall, acting like a valve, closes-off the side aperture preventing continued advancement or flow of the bone cement mixture through the side aperture. The wall further wipes the portion of the needle proximate the side aperture clean of bone cement residue, thus minimizing the likelihood of leaving bone cement remnants.

The side aperture's orientation may also facilitate breaking a bone cement bond formed between the needle and the bone in the event that the bone cement mixture solidifies prior to the needle being retracted through the bone opening. Specifically, when the needle is pulled and/or twisted, a concentration of shear stresses may be produced by edges in the cannula defining the side aperture moving against the solidified bone cement. These stresses result from the transverse orientation of the side aperture in relation to the axis of motion, that of pulling and twisting being along or about a longitudinal axis of the needle. This concentration of shear stresses may reduce the effort required to break the bone cement bond.

The needle is also configured to direct advancement of the bone cement mixture through the side aperture. Because of the highly viscous nature of the bone cement mixture, dispensing the mixture through an aperture transverse to the direction of flow in the needle may be more difficult. However, Applicant has discovered that by orienting an internal surface of the needle proximate the side aperture to direct flow of the bone cement mixture through the side aperture, dispensing and flowability of the mixture may be facilitated.

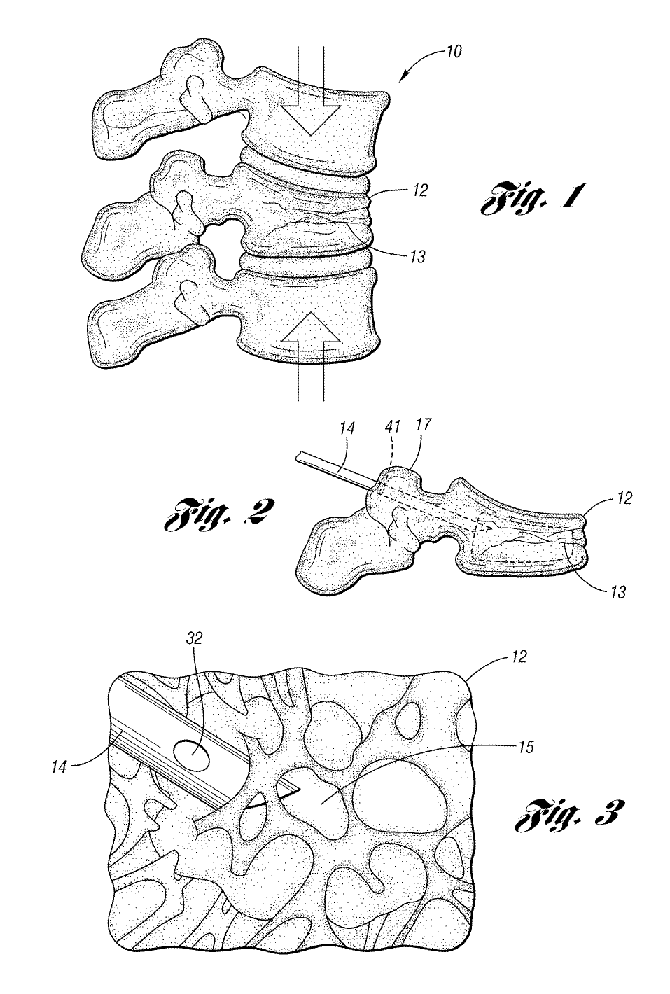

Referring now to the drawings, FIG. 1 illustrates a vertebra 10 which includes a collapsed vertebra 12 with a compression fracture 13. The vertebra 10 may be for example in the thoracic or lower spine of the patient. In the compression fracture 13 of the vertebra 12, the bone tissue of the vertebral body collapses. This condition is commonly caused by osteoporosis and less often by tumor, or trauma to the back.

Referring now to FIGS. 2 and 3, at least one embodiment of the present invention is provided. The collapsed vertebra 12 may be stabilized by either vertebroplasty or kyphoplasty, both of which are medical procedures for restoring structural integrity to the collapsed vertebra 12. These procedures stabilize the collapsed vertebra 12 by filling in open spaces 15 within the vertebra 12 to provide a more continuous and solid form. Kyphoplasty may further stabilize the vertebra 12 by restoring vertebral spacing which alleviates nerve pinching from the vertebra 12. It should be noted that the present invention applies to both of these medical procedures and other procedures for stabilizing and/or repairing the damaged bone of patients despite many of the various embodiments discussed herein as describing using vertebroplasty.

Vertebroplasty requires that the patient remain in a prone position throughout the entire procedure. It is performed under local anesthesia and/or a light sedative. A small nick is then made in the skin near the spine and a needle 14 is inserted percutaneously. As illustrated in FIG. 3, the needle 14 may be inserted into the interior open spaces 15 of the vertebra 12, for example via or through the left or right pedicle 17 of the vertebra 12.

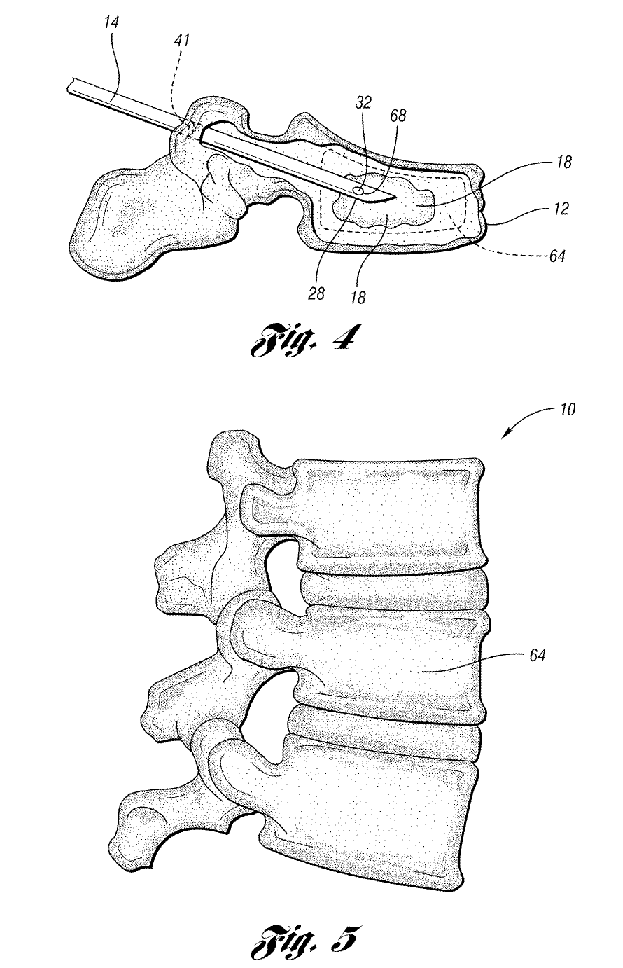

Referring to FIGS. 4 and 5, the bone cement mixture 18 may be dispensed from a syringe or other injection device through the needle 14 and into the vertebra 12 to form a solid structure 64 that supports the collapsed vertebra 12. The bone cement mixture 18 forms a solid structure 64 by chemically reacting or curing to become a solid. The stabilizing structure 64 may be formed within and/or about the collapsed vertebra 12 and may help restore vertebral spacing and alleviate nerve pinching by supporting the collapsed vertebra 12 generally in at least a compressive mode. Preferably, the structure 64 substantially fills in the open space 15 of the collapsed vertebra 12 providing a more dense and continuous vertebra 12 which enhances the mobility of, and alleviates pain in the patient.

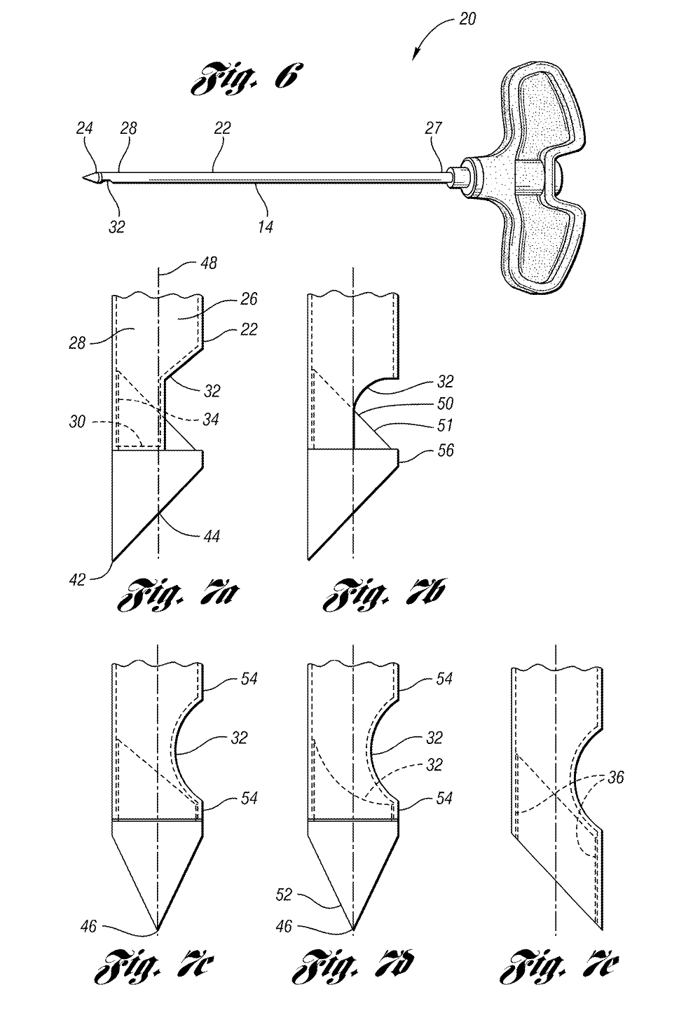

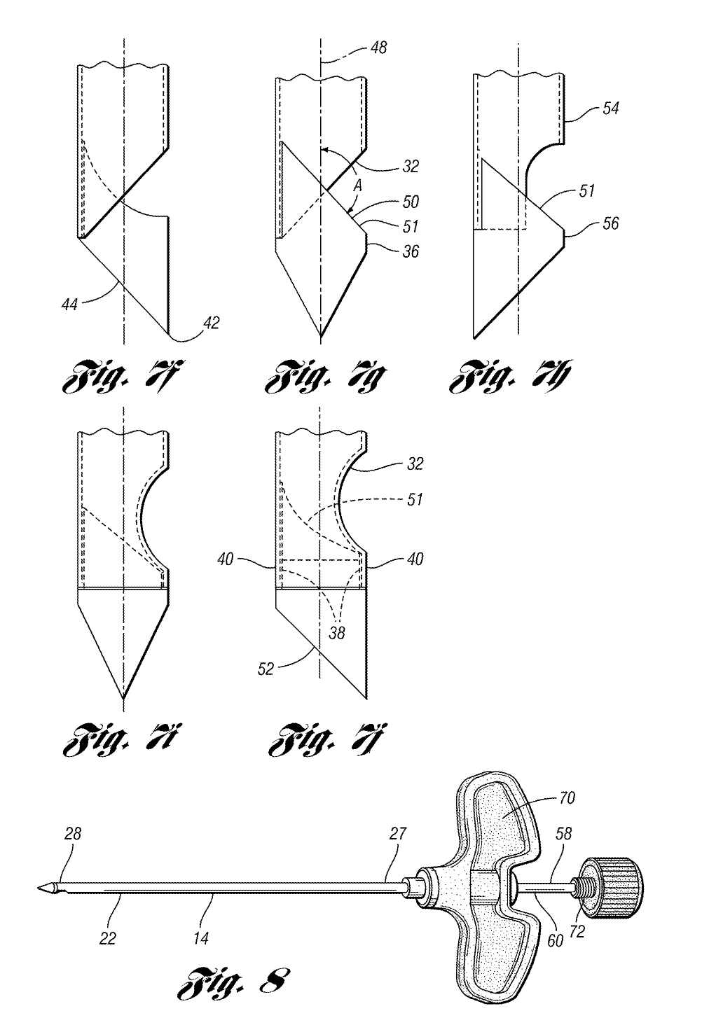

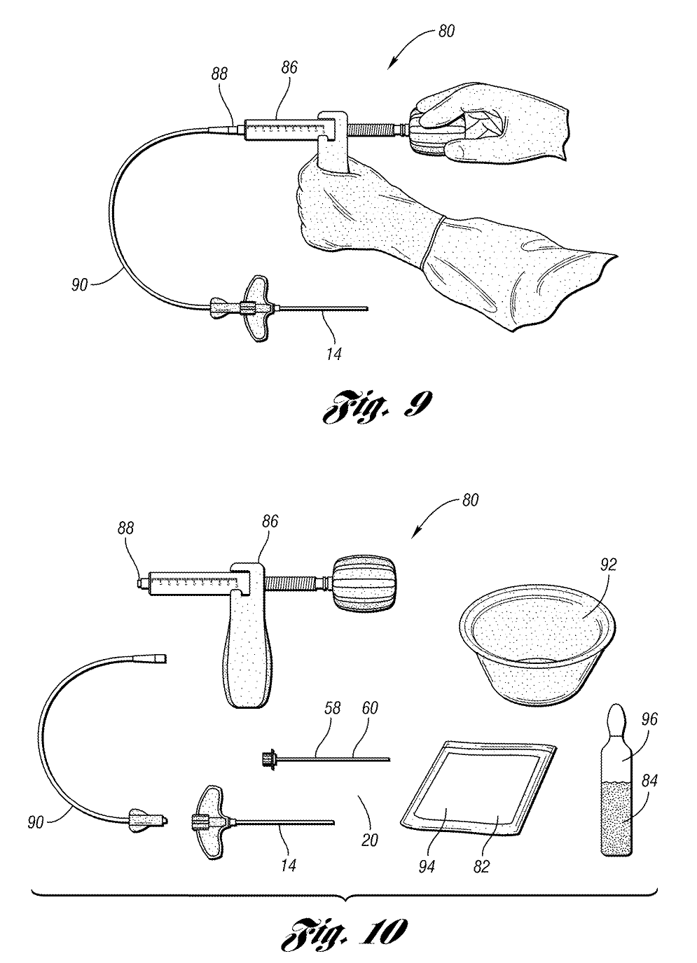

Referring to FIGS. 6-7j, at least one embodiment of a device 20 for introducing a bone cement mixture 18 into a damaged bone of a patient is provided. The device 20 comprises a needle 14. The needle 14 includes a cannula 22 and a tip portion 24 extending from the cannula 22. The cannula 22 may be straight or curved, or may flex between being straight and curved. The needle 14 and/or the cannula 22 and/or the tip portion 24 may be made of stainless steel, Nitinol, or any other suitable metallic or non-metallic material known to those skilled in the art.

The cannula 22 has a lumen 26 that is formed through the cannula 22. The cannula 22 has a proximal end 27 and a distal portion 28. The bone cement mixture 18 may be advanced through the lumen 26 from the proximal end 27 to the distal portion 28. The distal portion 28 has an open distal end 30 and a side aperture 32 formed through the distal portion 28.

The tip portion 24 is attached to the distal portion 28 of the cannula 22 such that the side aperture 32 is proximal the tip portion 24. The tip portion 24 may be integrally attached with the cannula 22 or separate and attached by, for example, welding, crimping, pressing, interference fit, threads or swaging. Alternatively, the tip portion 24 may be attached to the cannula 22 with an adhesive 34 and/or with solder 36.

The tip portion 24 may have at least one perimeter recess feature 38 formed therein and the distal portion 28 of the cannula 22 may include at least one extending feature 40 matched and aligned with the perimeter recess feature 38 for attaching and aligning the tip portion 24 to the distal portion 28. Adhesive 34 and/or solder 36 may be interposed between the perimeter recess feature 38 and the extending feature 40 to secure the tip portion 24 to the distal portion 28.

The tip portion 24 is configured for piercing the damaged bone 12 of the patient to define a bone opening 41 (see FIG. 4). In one example, the tip portion 24 has at least one beveled end 42 that has at least one edge 44 for piercing and cutting through the damaged bone 12 of the patient. Alternatively, the tip portion 24 may have a pointed end 46 that pierces and pushes through the damaged bone 12. For example, the collapsed vertebra 12 of a patient who is suffering from osteoporosis may be quite soft and the pointed end 46 may be adequate for penetrating and displacing the soft bone tissue, allowing the tip portion 24 to advance into the vertebra 12.

The tip portion 24 further directs advancement of the bone cement mixture 18 towards the side aperture 32 while preventing advancement of the bone cement mixture 18 through the distal end 30 of the distal portion 28. In at least one embodiment, the tip portion 24 is a solid, non-hollow structure that obstructs the open distal end 30, preventing flow of the bone cement mixture therethrough. The tip portion 24 has a longitudinal axis 48 and a proximal end 50. The proximal end 50 of the tip portion 24 has a surface 51 formed at an angle A to the longitudinal axis 48 which is between 90 and 180 degrees to define an obtuse angle. The surface 51 preferably faces towards the side aperture 32. The surface 51 may be substantially planar or curved. In one example, the tip portion 24 has a distal end 52 and the surface 51 is curved inwardly towards the distal end 52 of the tip portion 24.

The distal portion 28 is configured to tightly fit the bone opening 41 such that the advancement of the bone cement mixture 18 through the side aperture 32 is closed-off when the distal portion 28 is retracted through the bone opening 41. In one embodiment, the distal portion 28 has an outer perimeter 54 having cross-sectional dimensions substantially matching dimensions of the bone opening 41. Such as for example, if the bone opening 41 has a diameter of “X”, then the cross-section of the distal portion 28 will have a perimeter diameter of about “X”. In another embodiment, the tip portion 24 has an outer perimeter 56 and the surface 51 extends from the lumen 26 to the outer perimeter 56 where the outer perimeter 56 has cross-sectional dimensions substantially matching dimensions of the bone opening 41.

The surface 51 may be made, include or coated with a material containing a radiopacifier. The radiopacifier, which is detectable by fluoroscopic visualization, allows the interventionalist to monitor the position of the needle within the patient body during the medical procedure. Alternatively, the adhesive 34 and/or solder 36 may include a radiopacifier which also allows the interventionalist to monitor the procedure.

The distal portion 28 has edges 68 (shown in FIG. 4) defining the side aperture 32. The side aperture 32 may be circular, elliptical, slotted, squared or any other suitable shape for providing an exit opening for flow of the bone cement mixture from the needle 14 into the bone 12. In one example, the edges 68 may be beveled to facilitate cutting and/or breaking of a bone cement bond formed when the bone cement mixture cures prior to the needle 14 being retracted from the bone 12. The beveled edges 68 may sever the solidified bone cement mixture preferably by producing a concentration of shear stresses within the solid cement immediately adjacent to the edges 68 when the needle 14 is pulled and/or twisted.

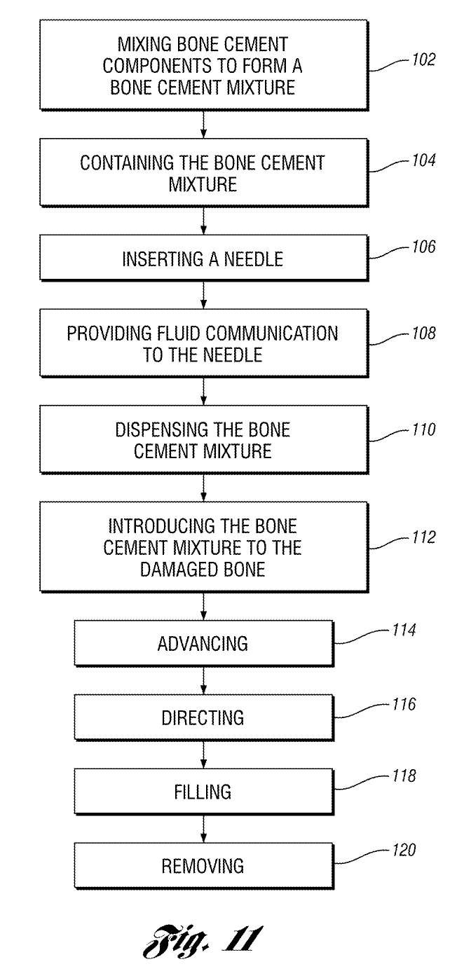

Referring to FIGS. 6, 8 and 10, the device may further comprise an obturator 58. The obturator 58 includes a shaft 60. The shaft 60 of the obturator 58 fits within the lumen 26 of the needle 14. The shaft 60 may be advanced through the lumen 26 towards the distal portion 28 of the cannula 22 so as to obstruct the side aperture 32 such that when the tip portion 24 pierces the damaged bone 12, the lumen 26 remains substantially free of fragments of the bone 12 or bone chips. Moreover, the obturator 58 reinforces the needle 14 when the shaft 60 is disposed through the lumen 26, providing structural integrity for inserting the needle 14 into the damaged bone 12.

In one embodiment, the needle 14 includes a hub 70 disposed adjacent to the proximal end 27 of the cannula. The obturator 58 includes a fastening member 72 attached to the shaft 60. The fastening member 72 may be selectively coupled to the hub 70 of the needle 14, for example, by threaded fastening, snap fitting or any other suitable removable fastening means known to those skilled in the art.

Referring to FIGS. 9 and 10, at least one embodiment of a bone cement substitute kit for introducing a bone cement mixture into the damaged bone of a patient is provided. The kit 80 includes the device 20 as discussed in the forgoing paragraphs as well as a first bone cement component 82 and a second bone cement component 84. In one example, the first bone cement component 82 is contained in an envelope 94 and the second bone cement component is contained in a glass ampoule 96. The first and second bone cement components 82 and 84 may be removed their respective containers 94 and 96 and mixed together within, for example, a mixing container 92 to form the bone cement mixture 18. In one embodiment, the first bone cement component comprises methylmethacrylate, sodium phosphate or a mixture thereof and the second bone cement component comprises polymethylmethacrylate, monocalcium phosphate, tricalcium phosphate, calcium carbonate or a mixture thereof.

An injection device 86 is for containing the bone cement mixture 18 and includes an outlet 88. In one example, the injection device 86 is a high pressure syringe capable of dispensing highly viscous bone cement mixtures having a viscosity substantially similar to a “paste.” The injection device 86 is configured to dispense the bone cement mixture 18 from the injection device 86 by advancing the bone cement mixture 18 through the outlet 88.

The kit 80 may further include a tubing 90. The tubing 90 may be coupled to the outlet 88 of the injection device 86 and the needle 14, providing fluid communication between the outlet 88 and the needle 14.

Referring to FIG. 11, a method for introducing a bone cement mixture into the damaged bone of a patient is provided. The method includes mixing a first bone cement component together with a second bone cement component to form a bone cement mixture 102. The bone cement mixture is contained in an injection device 104.

A needle is inserted 106 into the damaged bone of a patient. The needle includes a cannula and a tip portion extending from the cannula. The cannula has a lumen formed therethrough and a distal portion. The distal portion has an open distal end and a side aperture formed through the distal portion. The tip portion is attached to the distal portion of the cannula such that the side aperture is proximal the tip portion. Piercing the damaged bone to define a bone opening is the tip portion. The distal portion tightly fits through the bone opening.

The method further includes providing fluid communication between an outlet of an injection device and the lumen of the cannula 108. The bone cement mixture is dispensed through the outlet of the injection device 110.

The bone cement mixture is introduced into the damaged bone 112 of the patient, which includes advancing the bone cement mixture 114 through the lumen of the needle. Advancement of the bone cement mixture is directed through the side aperture 116 while preventing advancement of the bone cement mixture through the distal end of the needle via the tip portion. The damaged bone of the patient is filled with the bone cement mixture 118. The needle is then removed from the damaged bone 120 by at least one of a pulling motion and a twisting motion, wherein advancement of the bone cement mixture through the side aperture is closed-off when the distal portion is retracted through the bone opening.

As a person skilled in the art will readily appreciate, the above description is meant as an illustration of the implementation of the principles of this invention. This description is not intended to limit the scope or application of this invention in that the invention is susceptible to modification variation and change, without departing from the spirit of this invention, as defined in the following claims.

1. A device for introducing a bone cement mixture into a damaged bone of a patient, the device comprising:
a needle including a cannula and a tip portion extending therefrom, the cannula having a lumen formed therethrough and a distal portion, the lumen for advancing the bone cement mixture to the distal portion, the distal portion having an open distal end and a side aperture formed through the distal portion, the tip portion attached to the distal portion of the cannula such that the side aperture is proximal the tip portion, the tip portion configured for piercing the damaged bone of the patient to define a bone opening and to direct advancement of the bone cement mixture towards the side aperture while preventing advancement of the bone cement mixture through the distal end of the distal portion of the cannula, and the distal portion having an outer perimeter with cross-sectional dimensions substantially matching dimensions of the bone opening so as to tightly fit the bone opening such that advancement of the bone cement mixture through the side aperture is closed-off when the distal portion is retracted through the bone opening.

2. The device according to claim 1 wherein the tip portion has a longitudinal axis and a proximal end, the proximal end of the tip portion having a surface formed at an obtuse angle to the longitudinal axis facing towards the side aperture.

3. The device according to claim 2 wherein the tip portion has a distal end and the surface is curved inwardly towards the distal end of the tip portion.

4. The device according to claim 2 wherein the surface includes a radiopacifier detectable by fluoroscopic visualization.

5. The device according to claim 2 wherein the tip portion has an outside perimeter and the surface extends from the lumen to the outside perimeter of the tip portion and wherein the outside perimeter of the tip portion has cross-sectional dimensions substantially matching dimensions of the bone opening.

6. The device according to claim 1 wherein the tip portion is attached to the cannula with one of adhesive, solder, welding, crimping, swaging, interference fit and threads.

7. The device according to claim 6 wherein the one of the adhesive and the solder includes a radiopacifier detectable by fluoroscopic visualization.

8. The device according to claim 1 wherein the tip portion has at least one perimeter recessed feature formed therein and the distal portion of the cannula includes a least one extending feature matched and aligned with the perimeter recessed feature for attaching the tip portion to the distal portion of the cannula.

9. The device according to claim 1 wherein the tip portion has at least one beveled end having at least one edge for piercing the damaged bone of the patient.

10. The device according to claim 1 wherein the tip portion has a pointed end for piercing the damaged bone of the patient.

11. The device according to claim 1 wherein the distal portion of the cannula has a beveled edge that defines at least a portion of the side aperture.

12. The device according to claim 1 further comprising an obturator including a shaft, the obturator being configured to advance the shaft through the lumen towards the distal portion of the cannula so as to obstruct the side aperture such that when the tip portion pierces the damaged bone of the patient, the lumen remains substantially free of bone chips and wherein the obturator reinforces the needle when the shaft is disposed through the lumen.

13. The device according to claim 12 wherein the cannula has a proximal end, the needle including a hub disposed adjacent to the proximal end of the cannula and the obturator including a fastening member attached to the shaft, the fastening member being configured to selectively couple to the hub of the needle.

14. A bone cement substitute kit for introducing a bone cement mixture into the damaged bone of a patient, the kit comprising:
a first bone cement component and a second bone cement component, the first and second bone cement components for forming the bone cement mixture;
an injection device for containing the bone cement mixture and including an outlet, the injection device being configured to dispense the bone cement mixture from the injection device by advancing the bone cement mixture through the outlet; and
a needle in fluid communication with the outlet and including a cannula and a tip portion extending therefrom, the cannula having a lumen formed therethrough and a distal portion, the lumen for advancing the bone cement mixture to the distal portion, the distal portion having an open distal end and a side aperture formed through the distal portion, the tip portion attached to the distal portion of the cannula such that the side aperture is proximal the tip portion, the tip portion configured for piercing the damaged bone of the patient to define a bone opening and to direct advancement of the bone cement mixture towards the side aperture while preventing advancement of the bone cement mixture through the distal end of the distal portion of the cannula, and the distal portion having an outer perimeter with cross-sectional dimensions substantially matching dimensions of the bone opening so as to tightly fit the bone opening such that advancement of the bone cement mixture through the side aperture is closed-off when the distal portion is retracted through the bone opening.

15. The kit according to claim 14 further comprising a tubing configured to couple to both the outlet of the injection device and the needle and to provide fluid communication between the outlet and the needle.

16. The kit according to claim 14 further comprising an obturator including a shaft, the obturator being configured to advance the shaft through the lumen towards the distal portion of the cannula so as to obstruct the side aperture such that when the tip portion pierces the damaged bone of the patient, the lumen remains substantially free of bone chips and wherein the obturator reinforces the needle when the shaft is disposed through the lumen.

17. The kit according to claim 14 wherein the tip portion has a longitudinal axis and a proximal end, the proximal end of the tip portion having a surface formed at an obtuse angle to the longitudinal axis facing towards the side aperture.

18. A method for introducing a bone cement mixture into a damaged bone of a patient, the method comprising:
mixing a first bone cement component together with a second bone cement component to form the bone cement mixture;
containing the bone cement mixture in an injection device;
inserting a needle into the damaged bone of the patient, the needle including a cannula and a tip portion extending therefrom, the cannula having a lumen formed therethrough and a distal portion, the distal portion having an open distal end and a side aperture formed through the distal portion, the tip portion attached to the distal portion of the cannula such that the side aperture is proximal the tip portion, the tip portion piercing the damaged bone defining a bone opening, and the distal portion being configured to tightly fit the bone opening;
providing fluid communication between an outlet of the injection device and the lumen of the cannula;
dispensing the bone cement mixture from the injection device by advancing the bone cement mixture through the outlet; and
introducing the bone cement mixture to the damaged bone of the patient including:
advancing the bone cement mixture through a lumen of the needle;
directing advancement of the bone cement mixture through the side aperture while preventing advancement of the bone cement mixture through the distal end of the needle via the tip portion;
filling the damaged bone of the patient with the bone cement mixture; and
removing the needle from the damaged bone by at least one of a pulling motion and a twisting motion, wherein advancement of the bone cement mixture through the side aperture is closed-off when the distal portion is retracted through the bone opening.

19. The method according to claim 18 wherein the tip portion has a longitudinal axis and a proximal end, the proximal end of the tip portion having a surface formed at an obtuse angle to the longitudinal axis facing towards the side aperture.

20. The method according to claim 18 wherein the step of removing the needle includes shearing the bone cement mixture at an interface formed between the distal portion and the bone opening such that the bone cement mixture remains substantially within the damaged bone.
